(19) United States
(12) Patent Application Publication
(10) Pub. No.: US 2020/0026906 A1
(43) Pub. Date: Jan. 23, 2020

(54) FACE AUTHENTICATION DEVICE AND FACE AUTHENTICATION METHOD

(71) Applicant: MITSUMI ELECTRIC CO., LTD., Tokyo (JP)

(72) Inventors: Satoshi AJIKI, Yamagata-shi (JP); Toshiyuki TAKAHASHI, Higashine-shi (JP)

(21) Appl. No.: 16/499,840

(22) PCT Filed: Mar. 29, 2018
(86) PCT No.: PCT/JP2018/013468

§ 371 Date: Sep. 30, 2019

(30) Foreign Application Priority Data
Mar. 31, 2017 (JP) 2017-070293

(51) Int. Cl. G06K 9/00; G01B 11/22; G06T 7/00; A61B 5/1171

(52) U.S. Cl. CPC G06K 9/00281; G01B 11/22; G06T 2207/10028; A61B 5/1176; G06T 2207/30201; G06T 7/00

(57) ABSTRACT

A face authentication device 1 includes a three-dimensional face information storage part 7 storing at least one three-dimensional face information, an imaging optical system 3 for forming an optical image containing a face of an authentication target, an image sensor 5 for photographing the optical image of the authentication target to obtain two-dimensional image data and image plane phase differential information of the face of the authentication target and a face authentication processing part 9 for performing a three-dimensional face authentication for the authentication target by collating the face of the authentication target with the three-dimensional face information stored in the three-dimensional face information storage part 7 based on the two-dimensional image data and the image plane phase differential information obtained by the image sensor.

### TECHNICAL FIELD

The present invention generally relates to face authentication devices and face authentication methods, in particular to a face authentication device and a face authentication method which utilize an image sensor which can obtain two-dimensional image data and imaging plane phase differential information of a face of an authentication target.

### BACKGROUND ART

Generally, a variety of devices containing a mobile phone, a smart phone, a notebook computer and a laptop computer have utilized a combination of a password and an ID, a biometric authentication technology such as a fingerprint authentication, a voiceprint authentication and an iris authentication or the like in order to perform identity confirmation. Particularly, as a camera module is embedded in a variety of devices due to downsizing and high performance of the camera module in recent years, a face authentication technology which can perform the identity confirmation by photographing the face of the authentication target and then collating a photographed face image with a pre-registered face image of the person to be authenticated has been widely used.

Such an authentication method utilizing the face image of the authentication target has a “identity-fraud” problem that a third person impersonates the person to be authenticated by any means to fraudulently pass the authentication. For example, the third person often performs “identity-fraud” by making an authentication device photograph a face photograph of the person to be authenticated for fraudulent access when the face of the authentication target is photographed. In the identity-fraud utilizing the face photograph of the person to be authenticated, a face photograph printed on a media such as paper or a face photograph displayed on a displaying device such as a monitor is used.

Further, in the authentication method utilizing the face image of the authentication target, there is a possibility that an accuracy of the authentication decreases due to the presence or absence of cosmetic, changes in facial expression, directions of face, differences of illumination at the time of photographing or the like.

For solving these problems, a three-dimensional face authentication technology utilizing three-dimensional information on the face of the authentication target (three-dimensional face information) has been used. For example, patent document 1 discloses a face authentication device 500 as shown in FIG. 1, which includes a photographing part 501 for obtaining two-dimensional image data of a face of an authentication target and a plurality of distance sensors 502 for obtaining depth information of the face of the authentication target.

The face authentication device 500 of the patent document 1 focuses on the fact that the face photograph printed on the media such as paper and the face photograph displayed on the displaying device such as a monitor are planate. By utilizing this fact, the face authentication device 500 prevents the identity-fraud problem using the face photograph by determining whether a photographed object is planate or three-dimensional by using the depth information of the face of the authentication target obtained by the plurality of distance sensors 502 in addition to an ordinary face authentication using the two-dimensional image data obtained by the photographing part 501.

Further, the depth information of the face of the authentication target as described above does not change or slightly changes depending on the presence or absence of cosmetic, the changes in facial expression, the directions of face, the differences of illumination at the time of photographing and the like. Thus, it becomes possible to perform the face authentication with higher accuracy by performing the three-dimensional face authentication using the depth information of the face of the authentication target.

However, the face authentication device as disclosed in the patent document 1 needs to use the plurality of distance sensors for obtaining the depth information of the face of the authentication target in addition to the photographing part for obtaining the two-dimensional image date of the face of the authentication target. This results in a scale expansion of a system configuration, which causes some problems that the face authentication device gets larger, a power consumption amount of the face authentication device increases and a cost of the face authentication device increases.

Further, since the photographing part for obtaining the two-dimensional image data of the face of the authentication target and the plurality of distance sensors for obtaining the depth information of the face of the authentication target are provided at different positions, there is a disparity between the two-dimensional image data of the face of the authentication target and the depth information of the face of the authentication target obtained by the distance sensors. Thus, at the time of creating the three-dimensional face information of the authentication target by associating the depth information of the face of the authentication target with the two-dimensional image date of the face of the authentication target, it is required to perform a fitting process. Such a fitting process causes some problems that an accuracy of the face authentication decreases, a process time required for a face authentication process increases and a power consumption amount increases.

### RELATED ART

Patent document 1: JP 2006-259931A

### SUMMARY

### Problems to be Solved by the Invention

The present invention has been made in view of the conventional problems mentioned above. Accordingly, it is an object of the present invention to provide a face authentication device and a face authentication method which can prevent an identity-fraud problem using a photograph and achieve downsizing, power saving, cost reduction, high accuracy and speed-up of the face authentication device by utilizing an image sensor which can obtain two-dimensional image data and imaging plane phase differential information of a face of an authentication target.

### Means for Solving the Problems

The above object is achieved by the present inventions defined in the following (1) to (6).

(1) A face authentication device, comprising:

a three-dimensional face information storage part storing at least one three-dimensional face information;
an imaging optical system for forming an optical image containing a face of an authentication target;
an image sensor for photographing the optical image of the authentication target to obtain two-dimensional image data and imaging plane phase differential information of the face of the authentication target; and
a face authentication processing part for performing a three-dimensional face authentication for the authentication target by collating the face of the authentication target with the three-dimensional face information stored in the three-dimensional face information storage part based on the two-dimensional image data and the imaging plane phase differential information obtained by the image sensor.

(2) The face authentication device according to the above (1), wherein the face authentication processing part further performs a two-dimensional face authentication for the authentication target by using the two-dimensional image data of the face of the authentication target and outputs a signal indicating that a face authentication for the authentication target is successful when both of the two-dimensional face authentication and the three-dimensional authentication for the authentication target are successful.

(3) The face authentication device according to the above (1) or (2), further comprises a three-dimensional face information creating part for creating three-dimensional face information of the authentication target from the two-dimensional image data and the imaging plane phase differential information,

wherein the face authentication processing part performs the three-dimensional face authentication for the authentication target based on the three-dimensional face information of the authentication target.

(4) The face authentication device according to the above (3), wherein the face authentication processing part performs the three-dimensional face authentication for the authentication target by extracting a three-dimensional feature of the face of the authentication target from the three-dimensional face information of the authentication target and collating the extracted three-dimensional feature with a three-dimensional feature extracted from the three-dimensional information stored in the three-dimensional face information storage part.

(5) The face authentication device according to the above (4), wherein the three-dimensional feature contains at least one of a height of a nose, a shape of the nose, a depth of a hollow around an eye and distances between each part of a face in a depth direction.

(6) A face authentication method for performing a face authentication for an authentication target, comprising:

photographing, by an image sensor which can obtain two-dimensional image data and imaging plane phase differential information of a face of the authentication target, an optical image of the authentication target to obtain the two-dimensional image data and the imaging plane phase differential information of the authentication target; and
performing, by a processor, a three-dimensional face authentication for the authentication target by collating the face of the authentication target with three-dimensional face information stored in a three-dimensional face information storage part based on the two-dimensional image data and the imaging plane phase differential information of the authentication target.

### Effects of the Invention

The face authentication device and the face authentication method of the present invention use an image sensor which can obtain two-dimensional image data and imaging plane phase differential information of a face of an authentication target. Thus, the face authentication device and the face authentication method of the present invention can perform a three-dimensional face authentication with utilizing three-dimensional information of the face of the authentication target (three-dimensional face information) to prevent an identity-fraud problem using a photograph. Further, since the face authentication device and the face authentication method of the present invention can prevent an accuracy of a face authentication from decreasing due to the presence or absence of cosmetic, changes in facial expression, directions of face, differences of illumination at the time of photographing and the like, it is possible to improve the accuracy of the face authentication.

Further, for the face authentication device, it is not required to separately provide distance sensors for obtaining depth information of a face of a photographed object in addition to an image sensor for obtaining two-dimensional image data of the face of the photographed object unlike conventional face authentication devices. Thus, it is possible to achieve downsizing, power saving and cost reduction of the face authentication device.

Further, the image sensor used in the face authentication device and the face authentication method of the present invention can obtain both of the two-dimensional image data and the imaging plane phase differential information of the face of the authentication target. Thus, there is no disparity between the two-dimensional image data and the imaging plane phase differential information obtained by this image sensor. Therefore, it is unnecessary to perform a fitting process which has been performed in conventional face authentication devices and conventional face authentication methods. For this reason, the face authentication device and the face authentication method of the present invention do not have some problems that the accuracy of the face authentication decreases, a process time required for a face authentication process increases and a power consumption amount increases due to the fitting process.

### BRIEF DESCRIPTION OF THE DRAWINGS

FIG. 1 is a view showing a configuration of a conventional face authentication device.

FIG. 2 is a block diagram schematically showing a face authentication device of the present invention.

FIG. 3 is a flow chart for showing a face authentication method of the present invention.

FIG. 4 is a flow chart showing a face authentication process in the face authentication method shown in FIG. 3.

FIG. 5 is a view showing one of examples of application of the face authentication device of the present invention in a mobile device.

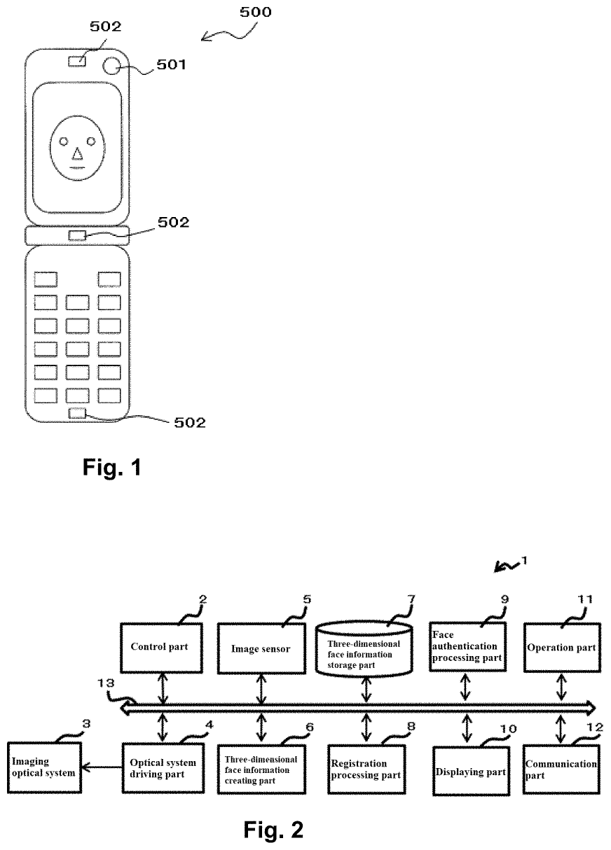

### DETAILED DESCRIPTION

Hereinafter, description will be given to a face authentication device and a face authentication method of the present invention based on a preferred embodiment shown in the accompanying drawings. First, the face authentication device of the present invention will be described in detail with reference to FIG. 2. FIG. 2 is a block diagram schematically showing the face authentication device of the present invention.

A face authentication method 1 of the present invention shown in FIG. 2 contains a control part 2 for controlling the face authentication device 1, an imaging optical system 3 for forming an optical image containing a face of an authentication target, an optical system driving part 4 for driving the imaging optical system 3 for providing an automatic focus function, a zoom function or the like, an image sensor 5 which can obtain two-dimensional image data and imaging plane phase differential information of the face of the authentication target, a three-dimensional information creating part 6 for creating three-dimensional face information of the authentication target from the two-dimensional image data and the imaging plane phase differential information obtained by the image sensor 5, a three-dimensional face information storage part 7 storing the three-dimensional face information, a registration processing part 8 for registering the three-dimensional face information in the three-dimensional face information storage part 7, a face authentication processing part 9 for performing a face authentication for the authentication target, a displaying part 10 such as a liquid crystal panel for displaying arbitrary information, an operation part 11 for inputting user operation, a communication part 12 for performing communication with external devices and a data bus 13 for transmitting and receiving data between respective components of the face authentication device 1.

The control part 2 transmits and receives various data and/or various instructions among the components through the data bus 13 to perform control of the face authentication device 1. The control part 2 includes a processor for performing operational processes and a memory storing data, programs, modules and the like required for performing the control of the face authentication device 1. The processor of the control part 2 uses the data, the programs, the modules and the like stored in the memory to perform the control of the face authentication device 1. Further, the processor of the control part 2 can provide desired functions by using each component of the face authentication device 1. For example, the processor of the control part 2 can use the face authentication processing part 9 to perform the face authentication for the authentication target.

For example, the processor of the control part 2 is one or more operation units such as microprocessors, microcomputers, microcontrollers, digital signal processors (DSPs), central processing units (CPUs), memory control units (MCUs), graphic processing units (GPUs), state machines, logic circuitries, application specific integrated circuits (ASICs) and combinations thereof that can perform operational processes for manipulating signals or the like based on computer-readable instructions. Among other capabilities, the processor of the control part 2 is configured to fetch computer-readable instructions (such as data, programs and modules) stored in the memory of the control part 2 and execute signal control and signal manipulation.

The memory of the control part 2 is one or more removable or non-removable computer-readable media including volatile memories (such as RAMs, SRAMs and DRAMs), non-volatile memories (such as ROMs, EPROMs, EEPROMs, flash memories, hard disks, optical discs, CD-ROMs, digital versatile discs (DVDs), magnetic cassettes, magnetic tapes and magnetic disks) and combinations thereof.

The imaging optical system 3 has a function of forming the optical image containing the face of the authentication target. The imaging optical system 3 is constituted of one or more optical elements such as a lens, a diaphragm and the like and collects light entering from the authentication target to form the optical image containing the face of the authentication target on an imaging plane of the image sensor 5. Further, the imaging optical system 3 may optionally include at least one of a focus lens for providing the automatic focus function, a zooming lens for providing the zoom function and an image stabilization lens for providing an image stabilization function. Namely, the imaging optical system 3 may be configured so as to provide only the function of forming the optical image containing the face of the authentication target, configured so as to provide only the automatic focus function as an additional function in addition to the function of forming the optical image or configured so as to provide all of the automatic focus function, the zoom function and the image stabilization function as additional functions in addition to the function of forming the optical image. In the case where the imaging optical system 3 is configured so as to provide at least one of the automatic focus function, the zoom function and the image stabilization function, the focus lens, the zooming lens and/or the image stabilization lens of the imaging optical system 3 are operated and driven by the optical system driving part 4.

The optical driving part 4 has a function of providing the automatic focus function, the zoom function and/or the image stabilization function by driving the focus lens, the zoom lens and/or the image stabilization lens of the imaging optical system 3. For example, the optical system driving part 4 can adjust a focus of the imaging optical system 3 by moving the focus lens of the imaging optical system 3 along an optical axis direction, adjust a magnification of the imaging optical system 3 by moving the zoom lens of the imaging optical system 3 along the optical axis direction and correct camera shake by moving the image stabilization lens of the imaging optical system 3 along a direction perpendicular to the optical axis direction. The optical system driving part 4 is not particularly limited as long as it can drive the focus lens, the zoom lens and/or the image stabilization lens. For example, the optical system driving part 4 can be constituted of an actuator such as a DC motor, a stepping motor and a voice coil motor.

In this regard, although the face authentication device 1 includes the optical system driving part 4 in the aspect shown in the drawing, the present invention is not limited thereto. For example, the scope of the present invention contains an aspect in which the imaging optical system 3 does not contain the focus lens, the zoom lens nor the image stabilization lens and the face authentication device 1 does not include the optical driving part 4. In this case, since the imaging optical system 3 of the face authentication device 1 is a single focus optical system and does not provide the automatic focus function, the zoom function nor the image stabilization function, the optical system driving part 4 is omitted from the face authentication device 1.

The image sensor 5 has a function of obtaining the two-dimensional image data and the imaging plane phase differential information of the face of the authentication target by photographing the optical image containing the face of the authentication target formed by the imaging optical system 3. In this regard, the term of “imaging plane phase differential information” in the specification refers to information representing how much a plane from which light entering into an arbitrary pixel of the imaging plane of the image sensor 5 travels is shifted from a best focus plane of the imaging optical system 3 in the optical axis direction.

For example, when light from a plane coinciding with the best focus plane of the imaging optical system 3 enters into an arbitrary pixel of the image sensor 5, the imaging plane phase differential information corresponding to this pixel is “0”. When light from a plane shifted from the best focus plane of the imaging optical system 3 in a direction toward the imaging optical system 3 enters into an arbitrary pixel of the image sensor 5, the imaging plane phase differential information corresponding to this pixel has a positive value depending on a shift amount of the plane. On the other hand, when light from a plane shifted from the best focus plane of the imaging optical system 3 in a direction apart from the imaging optical system 3 enters into an arbitrary pixel of the image sensor 5, the imaging plane phase differential information corresponding to this pixel has a negative value depending on a shift amount of the plane.

Thus, the imaging plane phase differential information of the face of the authentication target represents a distance of each part of the face of the authentication target from the best focus plane of the imaging optical system 3, that is the depth information of the face of the authentication target. In this regard, the imaging plane phase differential information of the face of the authentication target may correspond to all pixels of the two-dimensional image data of the face of the authentication target or correspond to some pixels required for performing the three-dimensional face authentication for the authentication target. For example, the imaging plane phase differential information of the face of the authentication target may correspond to only several percent (for example, 3 percent) of all pixels of the two-dimensional image data of the face of the authentication target.

By using the image sensor 5 as described above, it is possible to obtain the two-dimensional image data of the face of the authentication target and the imaging plane phase differential information corresponding to the depth information of the face of the authentication target with one image sensor. Thus, for the face authentication device 1, it is unnecessary to separately provide distance sensors for obtaining depth information of a face of a photographed object in addition to an image sensor for obtaining two-dimensional image data of the face of the photographed object. Thus, it is possible to achieve downsizing, power saving and cost reduction of the face authentication device 1.

Further, there is no disparity between the two-dimensional image data and the imaging plane phase differential information of the face of the authentication target obtained by the image sensor 5. Thus, in the face authentication device 1 of the present invention, it is unnecessary to perform a fitting process which has been required to be performed in the conventional face authentication devices. This makes it possible to prevent some problems that an accuracy of the face authentication decreases, a process time required for a face authentication process increases and a power consumption amount increases due to the fitting process.

In this regard, although the imaging plane phase differential information of the face of the authentication target obtained by the image sensor 5 is used for the three-dimensional face authentication for the authentication target in the face authentication device 1 of the present invention, the imaging plane phase differential information may be utilized for providing the automatic focus function due to the optical system driving part 4. In this case, the optical system driving part 4 moves the focus lens of the imaging optical system 3 along the optical axis direction so as to set the imaging plane phase differential information corresponding to a pixel at the focus point to be “0”, that is so as to be a best focus.

The three-dimensional face information creating part 6 has a function of creating the three-dimensional face information of the authentication target based on the two-dimensional image data and the imaging plane phase differential information of the face of the authentication target obtained by the image sensor 5. If the imaging plane phase differential information corresponds to all pixels of the two-dimensional image data of the face of the authentication target at the time of obtaining the imaging plane phase differential information with the image sensor 5, the three-dimensional face information creating part 6 creates the three-dimensional face information by associating the imaging plane phase differential information with each of the pixels of the two-dimensional image data. If the imaging plane phase differential information corresponds to only some pixels of the two-dimensional image data of the face of the authentication target at the time of obtaining the imaging plane phase differential information with the image sensor 5, the three-dimensional face information creating part 6 creates the three-dimensional face information by interpolating the imaging plane phase differential information based on the imaging plane phase differential information corresponding to some pixels of the two-dimensional image data by using an interpolation technology such as a bilinear interpolation method, a bicubic interpolation method and a nearest neighbor interpolation method and then associating the interpolated imaging plane phase differential information with each of the pixels of the two-dimensional image data.

The three-dimensional face information created as described above is stored in the three-dimensional face information storage part 7 by the registration processing part 8 at the time of registering the authentication target in the face authentication device 1 and collated with at least one three-dimensional face information stored in the three-dimensional face information storage part 7 at the time of performing the authentication process for the authentication target.

The three-dimensional face information storage part 7 is an arbitrary non-volatile storage media (such as a hard disk and a flash memory) for storing the three-dimensional face information. The three-dimensional face information of the authentication target created by the three-dimensional face information creating part 6 is stored in the three-dimensional face information storage part 7 at the time of registering the authentication target in the face authentication device 1. At the time of performing the authentication process for the authentication target, the three-dimensional face information storage part 7 stores at least one three-dimensional face information.

Although the three-dimensional face information storage part 7 is provided in the face authentication device 1 in the aspect shown in the drawing, the present invention in not limited thereto. For example, the three-dimensional face information storage part 7 may be an external server or an external storage device connected to the face authentication device 1 through a variety of wired or wireless networks such as Internet, a local area network (LAN) and a wide area network (WAN). Further, in the case where the three-dimensional face information storage part 7 is the external server or the external storage device, one or more three-dimensional face information storage parts 7 may be shared among a plurality of face authentication devices 1.

The registration processing part 8 has a function of performing a process for registering the authentication target in the face authentication device 1. At the time of registering the authentication target, the registration processing part 8 stores the three-dimensional face information of the authentication target created by the three-dimensional face information creating part 6 in the three-dimensional face information storage part 7. Further, at the time of registering the authentication target, the registration processing part 8 associates arbitrary information related to the authentication target (such as a name, a gender, an age, a body height, an expiration date for the authentication) inputted into the face authentication device 1 via the operation part 11 with the three-dimensional face information of the authentication target and stores both of the three-dimensional face information and the arbitrary information of the authentication target in the three-dimensional face information storage part 7.

The face authentication processing part 9 has a function of performing the three-dimensional face authentication for the authentication target by collating the face of the authentication target with the at least one three-dimensional face information stored in the three-dimensional face information storage part 7 based on the two-dimensional image data and the imaging plane phase differential information (that is the three-dimensional face information) of the face of the authentication target obtained by the image sensor 5. In this regard, the term of “three-dimensional face authentication” used in the specification refers to a face authentication process utilizing three-dimensional information of the face of the authentication target (such as the three-dimensional face information of the present invention).

At the time of performing the three-dimensional face authentication for the authentication target, the face authentication processing part 9 extracts a three-dimensional feature of the face of the authentication target from the three-dimensional face information of the authentication target and extracts a three-dimensional feature of a face from the at least one three-dimensional face information stored in the three-dimensional face information storage part 7. Then, the face authentication processing part 9 performs the three-dimensional face authentication by collating the three-dimensional feature of the face of the authentication target with the three-dimensional feature of the face extracted from the at least one three-dimensional face information stored in the three-dimensional face information storage part 7.

Examples of the three-dimensional feature extracted from the three-dimensional face information contain a height of a nose, a shape of the nose (such as a direction or a shape of a tip of the nose, a shape or a height of a back of the nose and a shape of a nasal wing), a depth of a hollow around an eye and distances between each part (such as an eye, a nose, a mouth, an ear and an eyebrow) of a face in a depth direction. These three-dimensional features of the face as described above can be extracted by creating a three-dimensional grid and texture of the face from the three-dimensional face information and modeling the face as a three-dimensional model.

The face authentication processing part 9 can perform the three-dimensional face authentication for the authentication target by collating at least one three-dimensional feature of the face of the authentication target extracted as described above with at least one three-dimensional feature extracted from the three-dimensional face information stored in the three-dimensional face information storage part 7.

Further, the face authentication processing part 9 may be configured to perform a two-dimensional face authentication for the authentication target by using the two-dimensional image data contained in the three-dimension face information in addition to the three-dimensional face authentication described above. Specifically, the face authentication processing part 9 may perform the two-dimensional face authentication for the authentication target by collating the two-dimensional image data of the face of the authentication target with two-dimensional image data contained in the three-dimensional face information stored in the three-dimension face information storage part 7 by using an arbitrary two-dimensional face authentication algorithm such as an eigenface method, a linear discrimination analysis, a graph matching method, a frequency analyzing method, a neural network method and a Viola-Jones method. In this regard, the term of “two-dimensional face authentication” used in the specification refers to a face authentication process utilizing two-dimensional information of the face of the authentication target (such as the two-dimensional image data of the face of the present invention).

In this case, the face authentication processing part 9 performs both of the two-dimensional face authentication and the three-dimensional face authentication for the authentication target and outputs a signal indicating that the face authentication for the authentication target is successful when both of the two-dimensional face authentication and the three-dimensional face authentication for the authentication target are successful. With this configuration, it is possible to perform a double check with the two-dimensional face authentication and the three-dimensional face authentication and thus improve the accuracy of the face authentication.

Further, although an execution order of the two-dimensional face authentication and the three-dimensional face authentication by the face authentication processing part 9 is not particularly limited, it is preferred that the face authentication processing part 9 is configured to first perform the two-dimensional face authentication and then perform the three-dimensional face authentication only if the two-dimensional face authentication is successful. Generally, a processing speed of the two-dimensional face authentication using the two-dimensional image data is faster than a processing speed of the three-dimensional face authentication using the three-dimensional model of the face or the like. Thus, by first performing the two-dimensional face authentication to screen the three-dimensional face information stored in the three-dimensional face information storage part 7 and then performing the three-dimensional face authentication by using only the three-dimensional face information with which the two-dimensional authentication is successful, it is possible to reduce a time required for performing the face authentication with the double check of the two-dimensional facial authentication and the three-dimensional face authentication.

After the face authentication for the authentication target by the face authentication processing part 9 has finished, a signal or the like according to a result of the face authentication for the authentication target is transmitted to the processor of the control part 2 and the processor of the control part 2 performs a process according to the result of the face authentication for the authentication target. For example, the processor of the control part 2 releases a lock of an arbitrary device or permits an arbitrary application to start-up when the face authentication for the authentication target is successful. On the other hand, the processor of the control part 2 keeps the lock of the arbitrary device or does not permit the arbitrary application to start-up when the face authentication for the authentication target is failed.

The display part 10 is a panel type display part such as a liquid crystal display part. The display part 10 displays various information containing the two-dimensional image data of the authentication target obtained by the image sensor 5, information indicating whether the face authentication for the authentication target is successful or failed, information required for operating the face authentication device 1 or the like in the shape of characters or images in accordance with the control from the processor of the control part 2.

The operation part 11 is used for performing operations by the user of the face authentication device 1. The operation part 11 is not particularly limited as long as the user of the face authentication device 1 can use the operation part 11 to perform the operations. For example, it is possible to employ a mouse, a keyboard, a numeric keypad, a button, a dial, a lever, a touch panel or the like as the operation part 11. The operation part 11 transmits signals respectively corresponding to the operations from the user of the face authentication device 1 to the processor of the control part 2.

The communication part 12 has a function of inputting data into the face authentication device 1 and/or outputting data from the face authentication device 1 to external devices. The communication part 12 may be connected to a network such as Internet. In this case, the face authentication device 1 can use the communication part 12 to perform communication with external devices such as a web server and a data server provided outside the face authentication device 1.

As described above, the face authentication device 1 of the present invention uses the image sensor 5 which can obtain the two-dimensional image data and the imaging plane phase differential information of the face of the authentication target. Thus, the face authentication device 1 can perform the three-dimensional face authentication with utilizing the three-dimensional information of the face of the authentication target (the three-dimensional face information) to prevent the identity-fraud problem using the photograph. Since the three-dimensional information of the face of the authentication target does not change or slightly changes depending on the presence or absence of cosmetic, changes in facial expression, directions of face, differences of illumination at the time of photographing and the like, it is possible to improve the accuracy of the face authentication by using the three-dimensional information of the face of the authentication target.

Further, for the face authentication device, it is not required to separately provide distance sensors for obtaining the depth information of the face of the photographed object in addition to an image sensor for obtaining the two-dimensional image data of the face of the photographed object unlike conventional face authentication devices. Thus, it is possible to achieve downsizing, power saving and cost reduction of the face authentication device 1.

Further, the image sensor 5 used in the face authentication device 1 can obtain both of the two-dimensional image data and the imaging plane phase differential information of the face of the authentication target. Thus, there is no disparity between the two-dimensional image data and the imaging plane phase differential information obtained by the image sensor 5. Therefore, it is unnecessary to perform a fitting process which has been performed in conventional face authentication devices and face authentication methods. For this reason, the face authentication device 1 do not have some problems that the accuracy of the face authentication decreases, a process time required for a face authentication process increases and a power consumption amount increases due to the fitting process.

Due to these advantageous matters, the face authentication device 1 of the present invention can prevent the identity-fraud problem using the photograph, achieve high accuracy of the face authentication and achieve the downsizing, the power saving, the cost reduction, the high accuracy and the speed-up of the face authentication.

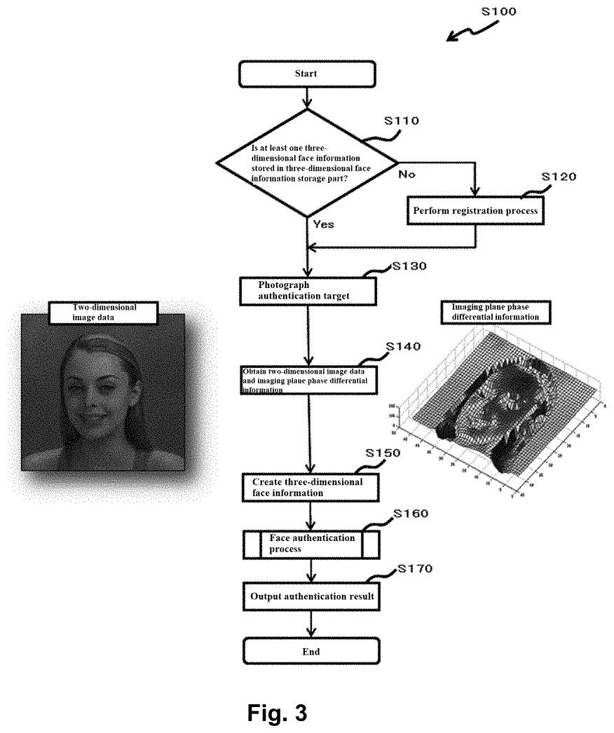

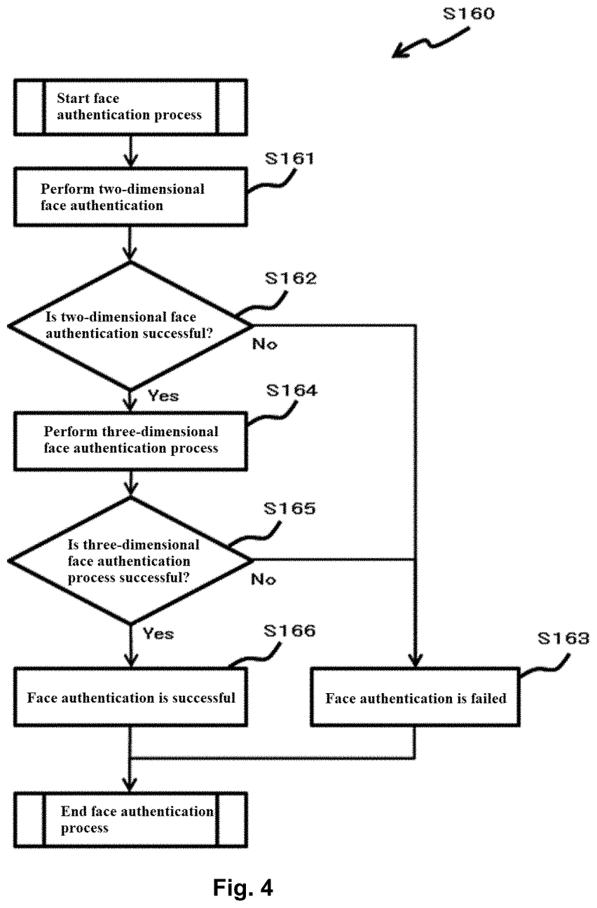

Next, description will be given to the face authentication method of the present invention with reference to FIG. 3 and FIG. 4. In this regard, although the face authentication method of the present invention can be performed with the face authentication device 1 of the present invention or an arbitrary device having a function equivalent to the function of the face authentication device 1 of the present invention, the following description will be given with assuming that the face authentication method of the present invention is performed with the face authentication device 1.

FIG. 3 is a flow chart for showing the face authentication method of the present invention. FIG. 4 is a flow chart showing a face authentication process in the face authentication method shown in FIG. 3.

A face authentication method S100 shown in FIG. 3 starts when the authentication target uses the operation part 11 for starting the face authentication process. At a step S110, it is determined whether or not at least one three-dimensional face information is stored in the three-dimensional face information storage part 7. In a case of determining that the at least one three-dimensional face information is not stored in the three-dimensional face information storage part 7 of the face authentication device 1, the process is shifted to a step S120.

At the step S120, a message for facilitating the authentication target to perform the registration process of the three-dimensional face information is displayed on the displaying part 10. After that, the authentication target operates the operation part 11 to start the registration process of the three-dimensional face information. When the registration process of the three-dimensional face information is started, the authentication target uses the imaging optical system 3, the optical system driving part 4 and the image sensor 5 to photograph his/her face. When the face of the authentication target is photographed, the two-dimensional image data and the imaging plane phase differential information of the face of the authentication target are obtained by the image sensor 5 and then the three-dimensional face information of the authentication target is created from the two-dimensional image data and the imaging plane phase differential information of the face of the authentication target by the three-dimensional face information creating part 6. After that, the registration processing part 8 stores the created three-dimensional face information of the authentication target in the three-dimensional face information storage part 7.

In this regard, the registration processing part 8 may perform a confirmation process with respect to the authentication target for confirming whether or not the three-dimensional face information of the authentication target should be registered in the three-dimensional face information storage part 7 before the registration process of the three-dimensional face information at the step S120 is started. For example, the registration processing part 8 facilitates the authentication target to input an ID and a password into the face authentication device 1 and confirms whether or not the authentication target has an administrative privilege for the face authentication device 1. In this case, the registration processing part 8 may perform the registration process of the three-dimensional face information at the step S120 only when it is confirmed that the authentication target has the administrative privilege for the face authentication device 1. On the other hand, when the registration processing part 8 does not confirm that the authentication target has the administrative privilege for the face authentication device 1, the face authentication method S100 finishes.

After the step S120 has finished or it is determined at the step S110 that the at least one three-dimensional face information has been stored in the three-dimensional face information storage part 7, the process is shifted to a step S130. At the step S130, a message for facilitating the authentication target to photograph his/her face of the authentication target with the imaging optical system 3, the optical system driving part 4 and the image sensor 5 is displayed on the displaying part 10. After the authentication target has used the imaging optical system 3, the optical system driving part 4 and the image sensor 5 to photograph his/her face, the process is shifted to a step S140.

At the step S140, the two-dimensional image data and the imaging plane phase differential information of the face of the authentication target are obtained by the image sensor 5. Next, at a step S150, the three-dimensional face information of the authentication target is created from the two-dimensional image data and the imaging plane phase differential information of the face of the authentication target by the three-dimensional face information creating part 6.

When the three-dimensional face information of the authentication target is created at the step S150, the process is shifted to a step S160. FIG. 4 is the flowchart for showing details of the step S160. When the step S160 is started, a two-dimensional face authentication process for the authentication target is performed by the face authentication processing part 9 at a step S161. At the step S161, the face authentication processing part 9 performs the two-dimensional face authentication process for the authentication target by collating the two-dimensional image data of the face of the authentication target contained in the three-dimensional face information of the authentication target with the two-dimensional image data of the face contained in the at least one three-dimensional face information stored in the three-dimensional face information storage part 7.

At a step S162, it is determined whether the two-dimensional face authentication process for the authentication target at the step S161 is successful or failed. In a case where two-dimensional image data to which the collation with the two-dimensional image data of the face of the authentication target is successful does not exist in the three-dimensional face information storage part 7 in the two-dimensional face authentication process at the step S161, it is determined that the two-dimensional face authentication process for the authentication target is failed. On the other hand, in a case where one or more two-dimensional image data to which the collation with the two-dimensional image data of the face of the authentication target is successful exists in the three-dimensional face information storage part 7, it is determined that the two-dimensional face authentication for the authentication target is successful.

In the case of determining that the two-dimensional face authentication process for the authentication target is failed at the step S162, the process is shifted to a step S163. At the step S163, the face authentication processing part 9 outputs a signal (message) indicating that the face authentication for the authentication target is failed to the processor of the control part 2 and then the step S160 finishes.

On the other hand, in the case of determining that the two-dimensional face authentication process for the authentication target is successful at the step S162, the process is shifted to a step S164. At the step S164, a three-dimensional authentication process for the authentication target is performed by the face authentication processing part 9.

At the step S164, the face authentication processing part 9 extracts the three-dimensional feature of the face of the authentication target based on the three-dimensional face information of the authentication target. Next, the face authentication processing part 9 performs the three-dimensional face authentication process for the authentication target for extracting the three-dimensional feature of the face from the three-dimensional face information which contains the two-dimensional image data to which the collation with the two-dimensional image data of the face of the authentication target is successful at the step S162 among the at least three-dimensional face information stored in the three-dimensional face information storage part 7 and then collating the three-dimensional feature of the face of the authentication target with the extracted three-dimensional feature from the three-dimensional face information stored in the three-dimensional face information storage part 7.

At a step S165, it is determined whether the three-dimensional face authentication process for the authentication target at the step S164 is successful or failed. In a case where a three-dimensional feature to which the collation with the three-dimensional feature of the authentication target is successful does not exist in the three-dimensional face authentication at the step S164, it is determined that the three-dimensional face authentication for the authentication target is failed. On the other hand, in a case where the three-dimensional feature to which the collation with the three-dimensional feature of the authentication target is successful exists, it is determined that the three-dimensional face authentication for the authentication target is successful.

In the case of determining that the three-dimensional face authentication process for the authentication target is failed at the step S165, the process is shifted to the step S163. On the other hand, in the case of determining that the three-dimensional face authentication process for the authentication target is successful at the step S165, the process is shifted to a step S166. At the step S166, the face authentication processing part 9 outputs a signal (message) indicating that the face authentication for the authentication target is successful to the processor of the control part 2 and then the step S160 finishes.

Referring back to FIG. 3, the processor of the control part 2 receives the signal from the face authentication processing part 9 and displays an authentication result according to the received signal on the displaying part 10 at a step S170. After that, the processor of the control part 2 performs a process according to the authentication result. For example, if the authentication result indicates that the face authentication is successful, the processor of the control part 2 performs a process for releasing a lock of an arbitrary device or permitting an arbitrary application to start-up. If the authentication result indicates that the face authentication is failed, the processor of the control part 2 performs a process for keeping the lock of the arbitrary device or not permitting the arbitrary application to start-up. When the authentication result is displayed on the displaying part 10 and the processor of the control part 2 performs the process according to the authentication result, the face authentication method S100 finishes.

Although the face authentication device and the face authentication method of the present invention have been described based on the embodiment shown in the accompanying drawings in the above description, the present invention is not limited thereto. The configuration of each component of the present invention may be possibly replaced with other arbitrary configurations having equivalent functions. Further, it may be also possible to add other arbitrary components to the configuration of the present invention.

For example, the number and the kind of the components of the face authentication device 1 shown in FIG. 2 are merely provided for the illustration of the present invention, the present invention is not necessarily limited thereto. The scope of the present invention contains alternations and changes of the described configuration in which arbitrary components are added or combined or arbitrary components are omitted without meaningfully departing from the principle and the spirit of the present invention. Further, each component of the face authentication device 1 may be practiced in the manner of hardware, in the manner of software or in the manner of the combination of hardware and software.

Further, the number and the kind of the steps of the face authentication method S100 shown in FIG. 3 and FIG. 4 are merely provided for the illustration of the present invention, the present invention is not necessarily limited thereto. The scope of the present invention contains alternations and changes of the described configuration in which arbitrary steps are added or combined or arbitrary steps are omitted without meaningfully departing from the principle and the spirit of the present invention.

For example, the two-dimensional face authentication process at the step S161 and the determination step at the step S162 shown in FIG. 4 may be omitted. Further, the execution order of the two-dimensional face authentication process and the three-dimensional face authentication process may be inversed.

### Examples of Application

As described above, since the face authentication device 1 of the present invention can prevent the identity-fraud problem using the photograph, achieve the high accuracy of the face authentication and achieve the downsizing, the power saving, the cost reduction, the high accuracy and the speed-up of the face authentication, it is possible to effectively utilize the face authentication device 1 in a variety of uses. Hereinafter, description will be given to examples of application of the face authentication device 1 of the present invention.

Mobile Device

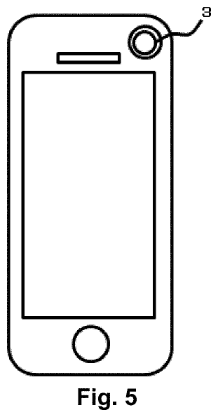

FIG. 5 is a view showing one of examples of application of the face authentication device of the present invention in a mobile device. As shown in FIG. 5, the face authentication device 1 of the present invention can be utilized in a mobile device such as a smartphone, a mobile phone, a PDA, a notebook computer, a mobile game machine, a mobile music player and a wearable device. In this case, the imaging optical system 3 of the face authentication device 1 is provided so as to be exposed from a front surface and/or a rear surface of the mobile device and each component of the face authentication device 1 containing the image sensor 5 is embedded in the mobile device. In this regard, all of the components of the face authentication device 1 may be embedded in the mobile device separately from components of the mobile device. Alternatively, some components of the face authentication device 1 may be practiced by the components of the mobile device. For example, the control part 2 of the face authentication device 1 may be alternatively practiced by a control part of the mobile device.

The mobile device as described above can perform a variety of processes depending on the result of the face authentication for the authentication target performed by using the face authentication device 1. For example, the mobile device may be configured to release a lock of the mobile device or permit an arbitrary application to start-up when the face authentication for the authentication target is successful.

As described above, by utilizing the face authentication device 1 in the mobile device, a user of the mobile device can perform identity confirmation only by photographing his/her face to release the lock of the mobile device or start-up the application. Thus, by utilizing the face authentication device 1 in the mobile device, it is possible to improve convenience of the mobile device.

Further, by utilizing the face authentication device 1 of the present invention, it is possible to solve the problems of the conventional two-dimensional face authentication such as the identity-fraud problem using the photograph and the decrease of the accuracy of the face authentication due to the cosmetic or the like. Thus, by utilizing the face authentication device 1 in the mobile device, it is possible to improve security of the mobile device.

Further, the three-dimensional face information for a plurality of persons may be stored in the three-dimensional face information storage part 7 of the face authentication device 1. In this case, it becomes possible to use the mobile device with a shared manner among the plurality of persons.

Fixed Device

The face authentication device 1 of the present invention can be utilized in a fixed device such as a laptop computer, a server, an ATM and a stationary game machine. In this case, the imaging optical system 3 of the face authentication device 1 is provided so as to be exposed from a front surface of the fixed device and each component of the face authentication device 1 containing the image sensor 5 is embedded in the fixed device. In this regard, all of the components of the face authentication device 1 may be embedded in the fixed device separately from components of the fixed device. Alternatively, some components of the face authentication device 1 may be practiced by the components of the fixed device.

As is the case with the above-mentioned example of application for the mobile device, the fixed device can perform a variety of processes depending on the result of the face authentication for the authentication target performed by using the face authentication device 1. For example, the fixed device may be configured to release a lock of the fixed device or permit an arbitrary application to start-up when the face authentication for the authentication target is successful.

As described above, by utilizing the face authentication device 1 in the fixed device, a user of the fixed device can perform identity confirmation only by photographing his/her face to release the lock of the mobile device or start-up the application. Thus, by utilizing the face authentication device 1 in the fixed device, it is possible to improve convenience of the fixed device.

Further, by utilizing the face authentication device 1 of the present invention, it is possible to solve the problems of the conventional two-dimensional face authentication such as the identity-fraud problem using the photograph and the decrease of the accuracy of the face authentication due to the cosmetic or the like. Thus, by utilizing the face authentication device 1 in the fixed device, it is possible to improve security of the fixed device.

System for Releasing Lock of Door

The face authentication device 1 of the present invention can be utilized in a system for releasing a lock of a door. In this case, the imaging optical system 3 of the face authentication device 1 is provided so as to be exposed from a front surface of the door, a wall or a ceiling near the door, or the like. The system for releasing the lock of the door as described above can perform a variety of processes depending on the result of the face authentication for the authentication target performed by using the face authentication device 1. For example, the system for releasing the lock of the door may be configured to release the lock of the door when the face authentication for the authentication target is successful.

In the case of using the system for releasing the lock of the door as described above for an entrance door of a house, the three-dimensional face information for all of family members is stored in the three-dimensional face information storage part 7 of the face authentication device 1. In the case of using the system for releasing the lock of the door as described above for a door of an office, the three-dimensional face information for all of workers or the like utilizing the office is stored in the three-dimensional face information storage part 7 of the face authentication device 1.

As described above, by utilizing the face authentication device 1 in the system for releasing the lock of the door, a user of a facility for which the system for releasing the lock of the door is utilized can release the lock of the door without using a key, an ID card or the like. Thus, it is possible to improve convenience of the facility for which the system for releasing the lock of the door is utilized.

Further, by utilizing the face authentication device 1 of the present invention, it is possible to solve the problems of the conventional two-dimensional face authentication such as the identity-fraud problem using the photograph and the decrease of the accuracy of the face authentication due to the cosmetic or the like. Thus, by utilizing the face authentication device 1 in the mobile device, it is possible to improve security of the facility for which the system for releasing the lock of the door is utilized.

In-Vehicle System

The face authentication device 1 of the present invention can be utilized in an in-vehicle system. In this case, the imaging optical system 3 of the face authentication device 1 is provided at one or more places in a vehicle so as to be exposed toward an inside of the vehicle. The in-vehicle system as described above can perform a variety of processes depending on the result of the face authentication for the authentication target performed by using the face authentication device 1. For example, the in-vehicle system may be configured to drive an engine of the vehicle when the face authentication for the authentication target sit in a driving seat of the vehicle is successful.

Further, in a case where a plurality of persons exist in the vehicle, the face authentication device 1 may perform the face authentication process for not only the person sitting in the driving seat but also the persons sitting in a passenger seat and a rear seat. With this configuration, it is possible to determine the number of the persons existing in the vehicle and whether or not the persons existing in the vehicle have been already registered in the face authentication device 1. For example, by providing the in-vehicle system as described above in a bus, it is possible to easily determine whether or not all of passengers who have been registered in the three-dimensional face information storage part 7 of the face authentication device 1 in advance and it is possible to easily confirm whether or not one or more registered passengers are replaced with other persons.

Although the examples of application of the face authentication device 1 of the present invention have been described in the above description, the application of the face authentication device 1 of the present invention is not limited thereto. It is possible to utilize the face authentication device 1 of the present invention for a variety of uses that a person having ordinary skill in the art can arrive.

In this regard, although the imaging plane phase differential information obtained by the image sensor 5 is mainly used for the three-dimensional face authentication for the authentication target in the face authentication device 1 and the face authentication method S100 of the present invention, an application method for the imaging plane phase differential information obtained by the image sensor 5 is not limited thereto. For example, by utilizing the imaging plane phase differential information obtained by the image sensor 5, it is possible to achieve a three-dimensional photographing for obtaining an image data to which the depth information (three-dimensional information) is imparted. Further, by utilizing the imaging plane phase differential information obtained by the image sensor 5, it is possible to perform length measurement photographing for measuring a distance from the photographed object.

### INDUSTRIAL APPLICABILITY

The face authentication device and the face authentication method of the present invention use the image sensor which can obtain the two-dimensional image data and the imaging plane phase differential information of the face of the authentication target. Thus, the face authentication device and the face authentication method of the present invention can perform the three-dimensional face authentication with utilizing three-dimensional information of the face of the authentication target (three-dimensional face information) to prevent the identity-fraud problem using the photograph. Further, since the face authentication device and the face authentication method of the present invention can prevent the accuracy of the face authentication from decreasing due to the presence or absence of cosmetic, the changes in facial expression, the directions of face, the differences of illumination at the time of photographing and the like, it is possible to improve the accuracy of the face authentication. Further, for the face authentication device, it is not required to separately provide distance sensors for obtaining depth information of the face of the photographed object in addition to an image sensor for obtaining two-dimensional image data of the face of the photographed object unlike conventional face authentication devices. Thus, it is possible to achieve the downsizing, the power saving and the cost reduction of the face authentication device. Further, the image sensor used in the face authentication device and the face authentication method of the present invention can obtain both of the two-dimensional image data and the imaging plane phase differential information of the face of the authentication target. Thus, there is no disparity between the two-dimensional image data and the imaging plane phase differential information obtained by the image sensor. Therefore, it is unnecessary to perform the fitting process which has been performed in the conventional face authentication devices and the conventional face authentication methods. For this reason, the face authentication device and the face authentication method of the present invention do not have some problems that the accuracy of the face authentication decreases, the process time required for the face authentication process increases and the power consumption amount increases due to the fitting process. For the reasons stated above, the present invention is industrially applicable.

1. A face authentication device, comprising:
a three-dimensional face information storage part storing at least one three-dimensional face information;
an imaging optical system for forming an optical image containing a face of an authentication target;
an image sensor for photographing the optical image of the authentication target to obtain two-dimensional image data and imaging plane phase differential information of the face of the authentication target; and
a face authentication processing part for performing a three-dimensional face authentication for the authentication target by collating the face of the authentication target with the three-dimensional face information stored in the three-dimensional face information storage part based on the two-dimensional image data and the imaging plane phase differential information obtained by the image sensor.

2. The face authentication device as claimed in claim 1, wherein the face authentication processing part further performs a two-dimensional face authentication for the authentication target by using the two-dimensional image data of the face of the authentication target and outputs a signal indicating that a face authentication for the authentication target is successful when both of the two-dimensional face authentication and the three-dimensional authentication for the authentication target are successful.

3. The face authentication device as claimed in claim 1, further comprises a three-dimensional face information creating part for creating three-dimensional face information of the authentication target from the two-dimensional image data and the imaging plane phase differential information,
wherein the face authentication processing part performs the three-dimensional face authentication for the authentication target based on the three-dimensional face information of the authentication target.

4. The face authentication device as claimed in claim 3, wherein the face authentication processing part performs the three-dimensional face authentication for the authentication target by extracting a three-dimensional feature of the face of the authentication target from the three-dimensional face information of the authentication target and collating the extracted three-dimensional feature with a three-dimensional feature extracted from the three-dimensional information stored in the three-dimensional face information storage part.

5. The face authentication device as claimed in claim 4, wherein the three-dimensional feature contains at least one of a height of a nose, a shape of the nose, a depth of a hollow around an eye and distances between each part of a face in a depth direction.

6. A face authentication method for performing a face authentication for an authentication target, comprising:
photographing, by an image sensor which can obtain two-dimensional image data and imaging plane phase differential information of a face of the authentication target, an optical image of the authentication target to obtain the two-dimensional image data and the imaging plane phase differential information of the authentication target; and
performing, by a processor, a three-dimensional face authentication for the authentication target by collating the face of the authentication target with three-dimensional face information stored in a three-dimensional face information storage part based on the two-dimensional image data and the imaging plane phase differential information of the authentication target.
